(19) United States
(12) Patent Application Publication
(10) Pub. No.: US 2005/0163833 A1
(43) Pub. Date: Jul. 28, 2005

(54) Production of starch-gel-based shaped bodies

(76) Inventor: Rolf Muller, Zurich (CH)

Correspondence Address: BACHMAN & LAPOINTE, P.C., 900 CHAPEL STREET, SUITE 1201, NEW HAVEN, CT, 06510, US

(21) Appl. No.: 10/493,453

(22) PCT Filed: Oct. 21, 2002
(86) PCT No.: PCT/CH02/00572

(30) Foreign Application Priority Data
Oct. 23, 2001 (DE) 101 52 125.1
May 6, 2002 (DE) 102 20 264.8

(51) Int. Cl. C08B 31/00; A61K 9/48

(52) U.S. Cl. 424/451; 106/206.1; 536/102

(57) ABSTRACT

Starch gels and a method for the production of shaped bodies therefrom containing active ingredients, especially gelatin-free soft capsules. Significant improvements, especially with regard to brittleness, storage stability in changing conditions and sorption behaviour, are provided in comparison with existing approaches to solving the gelatin problem in soft capsules. Gelatin-free soft capsules have resistant properties and reduced glyceamic index in comparison with starch or thermoplastic starch.

The present invention relates to a method for the production of shaped bodies, especially gelatin-free starch-gel-based soft capsules containing active ingredients.

### PRIOR ART

In accordance with the prior art, shaped bodies containing active ingredients such as soft capsules are still manufactured predominantly based on gelatin. However, as a result of the BSE problem, it has become urgent to replace gelatin for this application. Other disadvantages of gelatin are its animal origin and thus the non-acceptance of gelatin by vegetarians, vegans, Jews (since not kosher) and Muslims (pork gelatin).

The following requirements are primarily imposed on a gelatin replacement for this application: in order to be used for the production of soft capsules, especially by means of the rotary die method, a gelatin replacement must be able to be shaped into films having an elongation of at least 100% and a strength of at least 2 MPa under the processing conditions, which can be heat-sealed with themselves at temperatures below 100° C., preferably at the lowest possible temperatures, and which dissolve or break down in the stomach so that the active ingredients contained therein are released. In addition, the shaped and heat-sealed capsule must have good barrier properties with regard to the contents and good storage stability, i.e., they must show properties as constant as possible at temperatures in the range of 10 to 40° C. and air humidities in the range of 10 to 90%. In addition, the substances forming the soft capsule shell must be competitively priced in relation to gelatin.

Alternatives to gelatin have been developed so far but these have various disadvantages:

U.S. Pat. No. 5,342,626 describes the production of soft capsules by means of the rotary die method wherein the capsule shell is made of carrageenan, mannan gum, gellan or of mixtures of these plant polysaccharides. However, the mechanical properties of such capsules are unsatisfactory and the polysaccharides used are in some cases significantly more expensive compared with gelatin.

EP 0,397,819 describes a method for the production of thermoplastic starch having only a small crystalline fraction. However, the breaking elongation of corresponding films is significantly lower than required and the heat-sealability is problematical. In addition, the capsule properties show a marked dependence on the air humidity.

EP 0,542,155 describes thermoplastic starch and mixtures containing cellulose derivatives which again have inadequate mechanical properties and as a result of the high cost of the cellulose derivatives used, are only of limited competitiveness in this respect.

EP 1 103 254 A1 relates to a method for manufacturing a starch-containing shaped body and especially to the manufacture of a starch-containing, gelatin-free soft capsule. In this case, a mixture of starch, water and an organic plasticiser is melted and kneaded into a homogenised thermoplastic molten mass (thermoplastic starch, TPS). Then, if appropriate via an intermediate product in the form of a granulate, a film is extruded and this is then shaped into a shaped body, especially a soft-capsule half by means of the rotary die method.

EP 1 103 254 A1 contains no reference to a starch network or starch gel inside the soft capsule shell which in the sense of the teaching of the present invention is formed at least partly by heterocrystallisation and no process step is mentioned whereby a networking component is dissolved and mixed in this state with the basic starch. On page 5, lines 3 to 6 of EP 1 103 254 A1 a type of “starch network” is indeed mentioned but this is a network whose network points are constructed by intertwining and hooking as a result of the branchings of amylopectin macromolecules. A topological network is thereby claimed whereas the network in the sense of the teaching of the present invention is not formed by “loose” topological links but by “permanent” crystallites.

The shaped bodies manufactured according to the teaching of EP 1 103 254 A1 tend (a) on the one hand towards brittleness in an environment with little water (e.g. dry air in winter) since they readily release the softening water contained in them, (b) on the other hand, in an environment with copious amounts of water (e.g. moist air in summer) they tend to soften and thus tend to loose shape stability. This makes the TPS shaped bodies thus produced difficult to store and impedes their storage stability. With the technical teaching proposed in EP 1 103 254 A1 this can only be counter-controlled by increasing (a) or reducing (b) the plasticiser content.

However a shaped body which neither becomes more brittle or softens over a wide range of humidity cannot be produced thereby. In particular, the brittleness at low humidity has proved to be a serious obstacle to the commercialisation of the soft capsule according to EP 1 103 254 A1.

WO 99/02600 relates to a thermoplastic mixture of a biocatalytically produced poly-α-1,4-glucan (PAG), a thermoplastically processable polymer material, water in an amount sufficient for plasticisation and another plasticiser in addition to water. As explained on Page 18, lines 19 to 21, the components are mixed and processed to form TPS with the introduction of thermal and/or mechanical energy. This TPS can then be further processed into moulded parts such as films or hollow bodies.

Neither in the description nor in the examples (e.g. Examples 7, 8, 10, 13, 15, 16 and 17) is there any mention of a separate preparation of the components or any dissolution of networking or network-forming components and there is no reference relating to any physical network. In Example 15 (1 kg potato starch; 0.25 kg PAG, 0.3 kg glycerol, 1 g glyoxal as 40% aqueous solution), a cross-linking agent is certainly added with glyoxal but here glyoxal acts as a chemical cross-linking agent with the network points being obtained by covalent chemical bonds and not by crystallites in the sense of the teaching of the present invention.

It is certainly mentioned that biologically highly degradable mouldings with improved mechanical properties can be obtained but as in EP 1 103 254 A1, as a result of a lack of physical cross-linking, these mouldings have similar shortcomings such as sensitivity to water and humidity, brittleness and deficient shape stability.

A method for the production of thermoplastic-starch (TPS)-based soft capsules with a high softener content is described in WO 01/37817A1. However, the typical problematic properties of TPS which also stand in the way of a breakthrough for TPS as a biologically degradable plastic, namely their inherent brittleness and disadvantageous sorption behaviour, whereby the mechanical properties are strongly dependent on the air humidity, also have a disadvantageous effect regarding any use of TPS as a gelatin replacement in the area of soft capsules. This has the consequence that the storage stability of the corresponding capsules is unsatisfactory and even with high softener contents, they show a tendency to embrittlement and become friable, especially at low temperatures and low air humidity.

The production of soft capsules is described in WO 02/38132 A2, wherein a solution containing starch and at least one starch component having a reduced degree of branching compared with native starch is produced and then gelatinised. The gelatinisation is in this case primarily based on the starch having the reduced degree of branching. After gelatinisation has been completed, this starch gel is formed into a film by means of a shaping method and is then processed instead of gelatin film for the encapsulation using the rotary die method, wherein in each case the two capsule halves are formed, filled with active ingredient and heat-sealed. After the encapsulation has been completed, the capsules are dried. However, this solution has the following disadvantages: 1) Since the starch having the reduced degree of branching is dissolved together with other starches, wherein a large part of the solvent is required by these starches, a correspondingly reduced solvent fraction is available for the component having the reduced degree of branching whereby its solubility is drastically restricted. Thus, under the conditions specified in this unexamined laid-open patent application, only a weakly developed gel can be obtained. In order to improve the solubility of the starch having the reduced degree of branching and thus its subsequent gel formation, very high solvent concentrations must be used whereby however, the strength of the gel then formed is not sufficient for the rotary die method. 2) After the gel has been formed, a limited ductility exists as a result of the developed network structure. Whereas elongations of at least 100% are required for the rotary die method, only low elongations in the range of 10 to at most 40% can be obtained with starch gels, which are generally regarded as brittle in accordance with the prior art, whereby it is impossible to produce commonly used soft capsules such as oblong capsules, for example. The limited ductility of the gel films only allows very “flat” capsules having small internal volumes which are not accepted by the market. 3) Whereas softened TPS does allow some heat-sealing, this is only the case for starch gels to a limited extent and in particular, temperatures of at least 130° C. are required for this purpose (wherein barely adequate heat seals can be obtained at this impracticable temperature), i.e., at least partial dissolution of the network structure is required and for this reason, the production of heat-sealable marketable starch-gel-based soft capsules is not actually feasible in accordance with the proposed method. In relation to the further variants of starch gels mentioned in unexamined laid-open patent application WO 02/38132 A2 the afore-mentioned problems are even more marked.

Invention

As a result of the problems of the present technologies for the production of gelatin-free soft capsules, a new method is proposed whereby decisive advantages can be obtained compared with other proposed solutions. The method is based on the fact that, instead of a solution of starch and starch having a reduced degree of branching, a basic starch, which can be any starch, is plasticised by a thermoplastic method, a networking or gelatinisable starch or a mixture of such starches is dissolved separately, then added to the basic plasticised starch, mixed therewith preferably in a molecularly disperse fashion, wherein as a result of the mixing process there still exists a melt (and not a gel) which in a next step is formed into films or strips which can then be supplied to the rotary die method. Of fundamental importance here is that the gel or network formation is initiated after the film production at least partly before the encapsulation step whereby the good ductility of the plasticised starch and its heat-sealability are maintained. Network formation in significant fractions is only desired after encapsulation has been completed and then makes it possible to achieve the following advantages:
1) The onset of network formation after heat sealing has been completed makes it possible to achieve the ductility of at least 100% required for the rotary die method as well as an improvement in the sealing seam since this is strengthened by the network, which can be simply illustrated by the fact that the sealing seam is not only based on the hooking of starch macromolecules, as is the case with TPS, but is additionally based on a network formation wherein starch macromolecules each belonging to both halves of the soft capsules to be heat-sealed, are interlinked by microcrystallites which form the elastically active cross-linking points of the network.
2. A network formed after the encapsulation can be viewed as an elastic “internal reinforcement” whereby, on the one hand, the strength of the soft capsules is improved and on the other hand, its viscosity is also improved (reduced brittleness). By allowing a limited network formation during the forming of the capsules, an improvement in the melt strength of the plasticised starch and its ductility can also be achieved. Since, as a result of the stiffness of the macromolecules, starch only allows limited hooking of these macromolecules, whereby the melt strength and the strength of the sealing seam are also limited, a small partial network formation makes it possible to achieve an improvement with network elements formed by the microcrystallites complementing or taking over the function of the hooking points. In addition, elastic network elements are also more effective than hooking points, which especially in the case of rigid macromolecules only allow relatively loose fixing of macromolecules. According to a further aspect, the situation can also be considered such that, as a result of a small partial network formation before and/or during the forming of the resulting shaped bodies or soft capsules, a quasi-increased molecular weight is achieved by various macromolecules being physically linked together by network elements. This results in marked structurally viscous behaviour and increased melt strength and melt ductility. Since the kinetics of the network formation can easily be influenced by the method parameters and via the formulation, a limited network formation can easily be adjusted. An improvement in the viscosity (or reduction in the brittleness) of starch networks compared with TPS is obtained, on the one hand, by the elastic “internal reinforcement”, wherein however, this only applies to limited elongations up to a maximum of 40%, which however is more than adequate for finished soft capsules, and on the other hand, by the partial crystallinity of the network formed by microcrystallites if these microcrystallites are sufficiently finely distributed. The more finely distributed are the elastic network elements formed by the microcrystallites in the predominantly amorphous matrix, the greater are the positive effects of the network in relation to strength and viscosity. A highly disperse distribution of network elements is achieved by a high degree of dispersion of the solution containing networking starches which is mixed into the basic plasticised starch. The optimum is a molecularly disperse mixture which can be obtained, for example, by high shear velocities during the mixing process. A subsequent phase separation, whereby the mechanical properties would then deteriorate again, can be kinetically suppressed by means of suitable temperature control and by setting a high melt viscosity whereby the thermodynamically unstable state of a highly disperse mixture is frozen in. In particular, by means of molecular-disperse mixing it is possible to obtain single-phase starch gels and starch gels formed by heterocrystallisation, which are completely transparent and possess optimal mechanical properties.
3) The crystalline fractions show reduced water absorption compared with the amorphous fraction and thus a significantly more favourable sorption behaviour is obtained for starch gels compared with TPS which is almost completely amorphous, whereby the disadvantage of the marked brittleness of TPS soft capsules at lower air humidities can be countered by using starch gel. This advantageous effect is particularly marked if the microcrystallites can be obtained in the crystallographic “A” structural type since the A structural type can absorb only a fraction of water compared with the normally established “B” structural type which is the stable structural type at room temperature. The A structural type which is metastable at room temperature can be obtained and frozen in by suitable method parameters, especially by suitable temperature control or by subsequent heat treatment.
4) In addition to the various advantages for the production of soft capsules which are obtained by means of a network or gel formed at most partly during the shaping process, predominantly after the heat sealing, there is also the difference compared with TPS soft capsules, which dissolve in water or in gastric juice, that networks do not dissolve but swell in water and gastric juice. For the wall thicknesses and fractions of networking starches used for soft capsules the starch gel swells in water or gastric juice within minutes and thereby loses its strength almost completely whereby the capsules readily break down and the active ingredients contained in the soft capsules are released without any problems. At higher network densities, which can be set, for example by increased fractions of networking starches, the decomposition of starch gel soft capsules can be improved and accelerated by commonly used disintegrators such as calcium carbonate. The different breakdown behaviour of TPS and starch gel in an aqueous medium is important insofar as a unique and easy-to-verify differentiation between the two different types of capsule is hereby possible. At elevated network densities which can easily be obtained by the proposed method by increasing the fraction of networking starches, the degree of swelling of the resulting starch gel can be limited if required to such an extent that after swelling has taken place in water or in gastric juice, the resulting capsules have sufficient strength to prevent any breakdown. It is thus possible to achieve a retarded release of the active ingredients contained in the capsules by diffusion of said ingredients through the capsule shell swollen to a limited extent. Thus, controlled-release systems can also be produced on the basis of the proposed method for the production of soft capsules.
5) A further advantage of starch-gel-based soft capsules is that starch gel is prebiotic comparable to resistant starch. Despite its cost being many times higher than conventional starch, resistant starch is increasingly being added to various foodstuffs as an admixture because of its prebiotic effect. This effect is automatically obtained to a certain extent as a bonus with starch-gel-based soft capsules. As a result of the reduced glyceamic index compared with TPS soft capsules, soft capsules containing resistant fractions are also particularly advantageous for diabetics. It should also be mentioned that the prebiotic effect and also the reduction in the glyceamic index of starch gel is more marked compared with resistant starch. In addition to the absence of doubtful gelatin in starch-gel-based soft capsules, this is another important factor whereby the activity of such soft capsules can be increased more so, because not only the content of soft capsules but also the capsule shell itself is health-promoting. Among the various health-promoting effects of resistant starch, the stimulation of the immune system and the inhibition of colon cancer are especially topical.
6) By adding and mixing in a starch gel powder that has been specially optimised with regard to the prebiotic effect during the mixing process of basic starch and the solution of networking starch, the prebiotic effect of starch-gel-based soft capsule shells can be further increased and the glyceamic index further reduced. A further advantage can thereby be achieved as a result of the fact that the powdery prebiotic starch exhibits very severely limited water absorption whereby the sorption behaviour of soft capsules containing this component as a second phase can be further improved. Since this highly resistant second phase and the starch gel matrix are identical in terms of substance, but only differ in respect of different network densities, the phase coupling presents no problems.
7) Compared with WO 01/37817 A1, as a result of the partly crystalline or microcrystalline gel structure, smaller fractions of softeners such as water, sorbitol, maltitol or mannitol can be used so that the sorption behaviour and thus the product properties under changing air humidities can also be improved as a result of this effect.

All in all, compared with existing solutions in the area of gelatin-free soft capsules, a whole range of advantages are obtained with the proposed invention, relating to mechanical properties, heat-sealing and sorption behaviour, relating to a greater tolerance of the method, whereby a whole range of soft capsules having specifically optimised properties can be produced, and also in relation to new properties which had not yet been considered so far in the area of soft capsules, namely the starch-gel-based prebiotic effect and its reduced glyceamic index compared with starch or TPS, whereby the new technology opens up excellent market and marketing opportunities. This is all the more important in that the old gelatin technology in addition to the gelatin problem has become significantly less attractive because of cheap imitations.

The proposed method can be characterised in a simplified fashion by the fact that a basic starch or a mixture of basic starches, completely or partially plasticised, having a comparatively low softener content, is mixed in a molecular dispersed fashion with at least one completely or partly dissolved networking starch or with at least one completely or partly dissolved mixture of different networking starches having a comparatively high softener content. This is an important prerequisite especially for the formation of single-phase starch gels. Important process measures are overheating of the networking starch and if necessary, subsequent undercooling before the mixing process with the basic starch. By means of these two measures the temperature at which network formation is then initiated can be set to the desired range, and in particular it is possible to program the beginning of network formation such that partial network formation is present during the production step of the resulting shaped bodies or capsules, whereby the melt strength and melt ductility are improved but the heat sealing is not yet adversely affected however. The formation of the single-phase network structure is made possible by the choice of components whereby the molecular weights are of primary importance and by the kinetic control of the gelatinisation process by means of suitable method parameters. The mixture can then be formed into films which are supplied to the rotary die process.

Basic Starch

Any starch or any meal, as well as mixtures of various starches and/or meals can be used as basic starch. The basic starches can gelatinisable or not. The basic starch can be supplied to the method in any state, physically and/or chemically modified.

Examples of eligible basic starches or meals are those of the following origin: cereals such as maize, rice, wheat, rye, barley, millet, oats, spelt etc; roots and bulbs such as potato, sweet potato, tapioca (cassava), maranta (arrowroot), etc; pulses and seeds such as beans, peas, mungo, lotus etc. In addition, starches and meals of other origin are also eligible such as, for example, sago, yams etc. In addition, glycogen can also be used.

The starches can be modified by cultivation or genetic engineering methods such as, for example, waxy maize, waxy rice, waxy potato, high amylose maize, Indica rice, Japonica rice etc; they can have been modified by chemical methods such as, for example, by acid conversion, pyroconversion, cross-linking, acetylation, hydroxyethylation, hydroxypropylation, phosphorylation, graft reactions, reactions with amylases etc; they can have been modified by physical methods such as, for example, by gelatinisation (partly to completely), plasticisation, inhibition etc., or they can have been modified by a combination of cultivation, genetic methods, chemical and physical methods.

Examples of modified starches are thin-boiling starches, cold-water-soluble starches, pregelatinised starches, hydroxypropylated starches, dextrins, maltodextrin, limit dextrins, oligosaccharides, cationic starches, starch ether, starches obtained by fractionation.

Of particular interest are basic starches whose amylopectin fraction has an average chain length CL of at least 20, preferably of at least 22, more preferably of at least 24, most preferably of at least 26.

Furthermore of particular interest are basic starches whose amylopectin fraction has a blue value (BV) of at least 0.10, preferably of at least 0.13, more preferably of at least 0.16, most preferably of at least 0.18.

Also of particular interest are basic starches whose amylopectin fraction has an iodine affinity (IA) in g/100 g of at least 0.4, preferably of at least 0.6, more preferably of at least 0.8, most preferably of at least 1.0.

With respect to the molecular weight M_{w} (weight average) of basic starches, of particular interest are starches having a weight average of more than 10,000 g/mol, preferably of more than 50,000 g/mol, more preferably of more than 100,000 g/mol, most preferably of more than 500,000 g/mol.

Networking Starches

Networking starches can be defined in the following ways:
1. According to a first definition, these can be starches or meals which can form gels under suitable conditions. Exceptions therefrom are gels such as pure amylopectin gels which require very long gelatinisation times (days to weeks) and then form only very weak gels. Networking starches can be native or they can have been physically and/or chemically modified.
1A. One group of starches which satisfy this requirement are native or modified starches having an amylose content of at least 10%, preferably of at least 20%, more preferably of at least 30%, most preferably of at least 50%. Particularly suitable, for example, are high amylose starches, especially high amylose maize starches which can have an amylose content of up to approximately 100%, pea starches having amylose contents of more than 25% or amyloses of any origin.
1B. A further group of networking starches can be obtained by chemical and/or enzymatic decomposition, especially by debranching. Amylases such as alpha-amylase, beta-amylase, glucoamylase, alpha-glucosidase, exo-alpha-glucanase, cyclomaltodextrin, glucanotransferase, pullulunase, isoamylase, amylo-1,6-glucosidase or a combination of these amylases can be used for the enzymatic decomposition. Especially starches from the aforesaid group of starches can be used as starting materials for the decomposition. An example of chemical, non-enzymatic decomposition of starches is the hydrolysis by means of acids such as hydrochloric acid.
2. A further definition of networking starches relates to the degree of branching Q_{b}, wherein the degree of branching is less than 0.01, preferably less than 0.005, more preferably less than 0.002, most preferably less than 0.001, especially less than 0.0001.
3. Additionally designated as networking starches are predominantly linear starches which can crystallise after dissolution has taken place, but in the absence of further starches form not gels but dispersions of crystallites. Such starches have average degrees of polymerisation DP of typically less than 100, but in the presence of starches which can be both non-networking or also networking, can form gels by heterocrystallisation. Of interest in relation to this type of networking starch are starches having an average chain length CL or an average degree of polymerisation of at least 10, preferably of at least 20, more preferably of at least 30, most preferably of at least 50. In the case of starches, such a networking starch can, for example, be a debranched maltodextrin which cannot form any gel itself but with an amylopectin forms gels which are comparable to the amylose gels.
4. Networking starches can on the other hand also be characterised in that the macromolecules contain linear fractions wherein these linear fractions can be main or side chains having average degrees of polymerisation DP of more than 30, preferably more than 50, most preferably more than 80, especially more than 100, most especially more than 140.
5. In addition, a further group of networking starches can be obtained by fractionation of amylose-amylopectin mixtures, for example, by fractionation by means of differential alcohol precipitation, wherein the amylose and the intermediate fraction can be used as networking starch.

According to the invention, starches which satisfy at least one of conditions 1-5 are designated as networking starches. Also designated as networking starches are mixtures wherein the components and/or the mixture satisfy at least one of the above conditions.

It is noted that in certain cases, basic starch and networking starch can be identical in terms of substance since in principle, any networking starch can also be used as basic starch. Thus, the difference between basic starch and networking starch is not of a material type in all cases, rather the terms must also be defined in connection with the method. Networking starches are treated in such a way that their potential for forming networks is optimally released whereas this need not be the case with basic starch without a suitable dissolution and undercooling process.

Method

1. Dissolution and if Necessary Undercooling of Networking Starches

Only by suitably dissolving networking starches is their potential for forming networks released. As a result of plasticisation, as is commonly used for example in the production of thermoplastic starch, this is at most only partly ensured or at low softener concentration, very high temperatures are required which then lead to severe thermal decomposition. The dissolution process of networking starches is a multistage and complex process. The dissolution process usually extends over a temperature range of a few ° C. wherein successive order structures are dissolved, until complete dissolution has taken place. The temperature range is also strongly dependent on the concentration. The dissolution process is furthermore also dependent on any mechanical stressing by shearing, whereby dissolution can take place at lower temperature, and also on the pressure, dissolution time, heating rate and the pH.

However, overheating of the solution is preferred wherein a complete solution and thus standardisation is achieved. Overheating is understood as the process wherein a temperature higher than the solution temperature is applied. The nuclei effective for the network formation can then be obtained in a larger number and effectiveness by means of a defined undercooling whereby very finely structured networks with correspondingly good mechanical properties can be produced, especially single-phase gels. The various parameters of the dissolution and undercooling process are thus of central importance for the structure and properties of the gels obtained after the mixing process with basic starch.

Various networking starches can be dissolved together, undercooled and then mixed with basic starches. However, since different networking starches have different dissolution and nucleus formation characteristics, it is frequently logical to prepare them separately and supply them separately to the mixing process.

Since networking starches contain lipids and proteins which form complexes with the linear fractions of the networking starches and thus these linear fractions are no longer available for the network formation, in cases of higher lipid and protein fractions it is indicated that these substances are preliminarily removed by extraction. However, they can also be removed from the process by filtration after the dissolution process during the subsequent undercooling where they precipitate out from the solution. Preferably used are networking starches from root or bulb starches which only have negligibly small fractions of proteins.

The parameters of the dissolution and undercooling process are as follows:
1. The softener content WM_{d} in wt. % of the networking starches in step d) and e) lies in the range 40%-99%, preferably in the range 45%-97%, more preferably in the range 50%-95%, most preferably in the range 60%-92%.
2. The pressure p during the transfer in steps d) and e) is identical to the water vapour pressure p_{w}(T) at the respective temperature, preferably a maximum of 2 p_{w}(T), more preferably a maximum of 5 p_{w}(T), especially a maximum of 10 p_{w}(T), most preferably a maximum of 100 p_{w}(T).
3. The overheating temperature T_{L\overline{U}} is step d) is at least 120° C., preferably at least 140° C., more preferably at least 160° C., especially at least 180° C., most preferably at least 200° C. This temperature can be a maximum of up to 260° C., wherein such high temperatures can only be used for very short times. High temperatures T_{L\overline{U}} have a stabilising effect on the solution, i.e., the higher T_{L\overline{U}}, the lower the temperature at which the solution still remains stable afterwards, whereby the tolerance of the method is increased. High temperatures T_{L\overline{U}} are especially important to control the beginning of network formation after the solution has been mixed with the basic starch. The higher T_{L\overline{U}}, the lower the temperature at which the network formation then begins.
4. The duration delta t_{d} of the transfer in step d) is a maximum of 7 min, preferably a maximum of 3 min, more preferably a maximum of 1 min, especially a maximum of 0.5 min, most preferably a maximum of 0.2 min, and the minimum duration is 5 sec. Short transfer times are especially important at high temperatures T_{LU} in order to suppress thermal decomposition.
5. The heating rate v_{d} during transfer in step d) is at least 1° C./min, preferably at least 10° C./min, more preferably at least 50° C./min, especially at least 100° C./min, most preferably at least 200° C./min, and the maximum heating rate is approximately 300° C./min. High heating rates are especially important at high concentrations of networking starches, at high molecular weights of these starches, at high temperature T_{L\overline{U}} in step d) and to suppress thermal decomposition of networking starches.
6. The temperature T_{L1} in step e) is a maximum of 0.9 T_{L\overline{U}}, more preferably a maximum of 100° C., especially a maximum of 70° C., most preferably a maximum of 30° C. The minimum temperature is approximately 0° C. Low temperatures T_{L1} are important for setting high undercoolings and for setting high nuclei numbers. This is generally important for the production of high-strength networks with the lowest degrees of swelling since high degrees of swelling should be set for starch gel soft capsules if the undercooling of solutions containing networking starches is only of minor importance.
7. The duration delta tₑ of the transfer in step e) is a maximum of 7 min, preferably a maximum of 3 min, more preferably a maximum of 1 min, especially a maximum of 0.5 min, most preferably a maximum of 0.2 min, the shortest times are around 5 sec. Short times are important in connection with high-strength networks in order to obtain high undercoolings delta T_{LU} and thus high nuclei numbers Zₖ without any network formation or crystallisation of the networking starch being initiated. For starch gel soft capsules these parameters and effects are secondary.
8. The cooling rate vₑ during transfer in step e) is at least 5° C./min, preferably at least 30° C./min, more preferably at least 70° C./min, especially at least 110° C./min, most preferably at least 200° C./min and the maximum cooling rate is around 300° C./min. By means of high cooling rates a high nuclei number can be achieved in the second fluid Zₖ without any network formation or crystallisation of networking starches being initiated.
9. The pH in steps d) and e) for starch is in the range 5-12, preferably in the range 6-12, more preferably in the range 7 to 12. An elevated pH facilitates the solubility of networking starches. If necessary, the pH of the total mixture in step g) can be adjusted to the desired value, preferably to pH6-8, by adding a salt or base.
10. The shear velocity G_{d} in steps d) and/or e) and f) is at least 10/s, preferably at least 100/s, more preferably at least 1000/s, especially at least 10,000/s, most preferably at least 50,000/s. The maximum shear velocities are around 100,000/s. By means of high shear velocities the solubility especially of networking starches having a high molecular weight can be significantly improved in step d) and thus higher concentrations can be processed. In step e) high shear velocities prevent premature network formation.

Starches treated in accordance with the above conditions 1 to 10, are then mixed with basic starches to obtain networks wherein both networking starches and basic starches make a contribution to the forming network.

After a networking starch or a mixture of networking starches has been dissolved in accordance with the above conditions and undercooled if necessary, they can be mixed directly with the basic starch or however, two or a plurality of solutions are first brought together, mixed and then supplied to the basic starch. In certain cases, it is also possible to mix prepared networking starches into respectively different first fluids of basic starches and then combine these mixtures to form a total mixture.

2. Mixing Basic Starch with Networking Starch

A molecular disperse mixture of basic starch and networking starch is an important requirement especially to obtain single-phase gels. Such mixtures can be obtained by using shearing and high shear velocities. If a molecular disperse or almost molecular disperse mixture has been obtained, any phase separation can be limited or completely prevented by kinetic control of the process. This means corresponding control of the cooling rate wherein the single-phase thermodynamically metastable state can be frozen in.
1. The softener content WM₁ in wt. % in the basic starch in step c) before the supply of networking starch is 5%-90%, preferably 5%-70%, more preferably 5%-60%, especially 5%-50%, most preferably 5%-45%.
2. The average degree of branching Q_{b} of the starch mixture in step g) is usually higher than the average degree of branching of the networking starch used as a result of the mixing with generally significantly more strongly branched basic starches, and Q_{b} is less than 0.05, preferably less than 0.02, more preferably less than 0.006, especially less than 0.003, most preferably less than 0.001.
3. The softener content WMₛ in wt. % directly after step g) is less than 80%, preferably less than 75%, more preferably less than 70%, especially less than 65%, most preferably less than 60%. The minimum softener content WM₂ is 15%.
4. The shear velocity G_{g} during mixing of the first fluid with the second fluid is at least 10/s, preferably at least 100/s, more preferably at least 1000/s, especially at least 10,000/s, most preferably at least 50,000/s. The maximum shear velocity is around 100,000/s. By means of high shear velocities preferably a molecular dispersed mixture of fluids is achieved which is a requirement for high resulting network densities N₀/V₀ and especially for single-phase networks. In addition, as a result of high shear velocities G_{g} a large number of smallest possible crystallites forming the network elements is obtained.
5. In addition, the network density can be increased after network formation has taken place in the mixture by means of suitable foreign nucleating agents. The number of nuclei Z effective in the network formation is then given by Z=Zₖ+Z_{N}, where Zₖ is the number of nuclei in the second fluid and Z_{N} is the number of foreign nuclei.
6. The concentration of networking starch processed in accordance with steps d) to f) in the mixture of step g) in wt. % is 1-95%, preferably 2-70%, more preferably 3% to 50%, especially 3% to 30%, most preferably 3-25%. By using high concentrations of networking starches in the second and third fluid, correspondingly high concentrations of networking starches can be obtained in the mixture after mixing with basic starches, whereby high network densities and thus high network strengths can be obtained.

In at least one of steps a) to g) at least one softener can be at least partly removed from the process and this is especially important in step g) since the phase separation can be suppressed by reducing the softener content while restricting the mobility of the molecules.

3. Film Formation, Reforming and Network Formation

After the networking starches have been dispersed in a first fluid, the admixtures have been mixed in and the softener content WM₃ has been adjusted, and the mixture has reached the temperature T₃, a film is produced therefrom. The film can be divided in two in the longitudinal direction and the two halves can than be fed to a reforming plant wherein the two halves of the soft capsules produced, filled and heat-sealed therein come from the two film halves. Alternatively, two films at a time can also be produced in parallel, which are then fed to the reforming plant. The network formation is initiated shortly before or during the reforming into the resulting soft capsules by lowering the temperature. A further possibility for controlling the beginning of network formation consists in the selection and concentration of networking starches, wherein a wide tolerance is available in relation to the temperature at which the network formation or gelatinisation is initiated. A further possibility for influencing the gelatinisation temperatures consists in the choice of solution or overheating temperature, undercooling and further parameters of the process steps d) and e). During the reforming into the resulting soft capsules, wherein high elongations are used, the network formation must by no means be complete because this would inevitably result in tearing of the material. However, a small proportion of network formation, of a few percent with respect to the completely developed network, can be advantageous because the structural viscosity of the melt and thus its ductility is thereby improved. The process is controlled such that the network formation mainly takes place after the heat sealing of the soft capsule halves. After this time the fastest possible network formation is advantageous. This can be accelerated, for example, by the resulting soft capsules being briefly cooled in a cold air flow at low air humidity. As a result, the soft capsules gain in strength and their surface exhibits almost no stickiness as a result of the gelatinisation, whereby the further treatment of the capsules is simplified.

Gels or networks having low softener contents are transparent because the size of the crystallites is below the wavelength of visible light and the crystallites thus cannot scatter the light. This is an indication that it has been possible to obtain a very small crystallite size as a result of the measures taken. Such transparent gels are described as single-phase gels. At higher softener contents larger crystallites are formed whose size is of the order of magnitude of or greater than the wavelength of visible light, which can therefore scatter the light, are thus not transparent and have a milky white shade, as can be seen with conventional gels. However, the transparency is controlled not only via the softener content, but also decisive are the degree of dispersion of the solution of networking starch, its concentration in the total mixture, the viscosity and especially the parameters in steps d) and e).

Steps a) to k) are preferably carried out continuously, at least in part areas wherein the suitable process zone of the process space is at least one mixer and steps a) to g) take place continuously in successive sections of the at least one mixer and steps h) and i) takes place in a shaping or reforming unit following the at least one mixer. The at least one mixer can be a single-screw or a double-screw or a multiple-screw or a ring extruder or a co-kneader or a static mixer or a Ystral mixer or an agitator ball mill or another process stretch which is controllable with respect to temperature, pressure and shearing. Current thermoplastic shaping methods can be used to produce the film, for example, extrusion through a wide-slit nozzle followed by section rolling. The reforming plant is preferably a continuously operating encapsulation plant, for example a rotary die plant. In a further variant of the method, steps a) to c) are carried out preliminarily wherein granules of thermoplastic starch are obtained which can be transported and put into intermediate storage. The thermoplastic starch is then transferred again into a melt, whereupon this melt can be mixed in steps f) and g) with one or a plurality of solutions of networking starches which are prepared in accordance with steps d) and e).

No limits are imposed on the shape of the soft capsules, these can be any shape and in addition, two- and multi-chamber capsules can also be produced. Fillers in accordance with the prior art such as liquid to pasty substances, powder, beads, granules etc. can be used as filler. In addition to soft capsules, it is also possible to produce paint balls and further products which are produced using soft gelatin encapsulation techniques in accordance with the prior art. In addition, a layer of multilayer shaped bodies, especially of multilayer soft capsules, can be produced using the film produced by the proposed method. Further layers can consist of, for example, PEG, alginates, carrageenans or modified cellulose such as HPMC.

Softeners

The same solvents, softeners and softener mixtures which are suitable as solvents, softeners and softener mixtures for starch or thermoplastic starch according to the prior art, can be used as softeners, and these are preferably selected from the following group:
Water; glycerol; glycerol ethoxylate; polyglycerols; di-, to decaglycerols; polyglycerol monoethoxylates; reactions products of glucose with ethylene oxide; glucose monoethoxylate; glucoside; butylglucoside; alpha-methylglucoside; maltose, glucotri- and higher glucopolysaccharides, mono- and oligosaccharide syrups; alcohols; polyalcohols; butanol; erythritol; pentaerythritol; triethylolpropane; trimethylolpropane; triethylpropane monoethoxylate; propanediols; butanediols; pentanediols; hexanediols; hexanetriols; polyvinyl alcohols with 3 to 20 monomer units; polyvinyl acetates completely or partly hydrolysed to polyvinyl alcohols; trihydroxymethylaminomethane; amino alcohols; fatty alcohols; amines; hydroxyalkydamine; ethylenediamine; amides; esteramides; formamide; acid amides; sulfoxides; DMSO; quaternary ammonium compounds; glycol, ethylene glycol; ethylene diglycol; ethylene triglycol; propylene glycol; propylene diglycol; propylene triglycol; neopentyl glycol; polyethylene glycols; polypropylene glycol; polyglycols; pyrrolidone; 2-pyrrolidone or 1-methyl-2-pyrrolidone; caprolactam; polycaprolactam; sorbitol; sorbitol acetate; sorbitol diacetate; sorbitol monoethoxylate; sorbitol dipropoxylate; sorbitol diethoxylate; sorbitol hexaethoxylate; salts of carboxymethyl sorbitol; aminosorbitol; maltitol; mannitol; mannitol monoacetate; mannitol monoethoxylate; xylitol; arabitol; adonitol; iditol; galactitol; allitol; acids; carboxylic acids; formic acid; acetic acid; succinic acid; succinic acid anhydride; adipinic acid; lactic acid; tartaric acid; citric acid: malic acid; hydroxybutyric acid; maleic acid; fatty acids; dimethylsulfoxide; urea; chemically modified elements of this group, especially obtained by esterification; mixtures of elements of this group.

Softeners or softener mixtures are usually supplied to the basic starches in step b) and to the networking starches in step d), additional softener can also be supplied to the method in at least one of steps a), c), e), f) or g). The supply of softener in step b) can be dispensed with wherein the step c) is also dispensed with and the corresponding basic starch is transferred into a fluid or plasticised in step g) at the same time as mixing to the total mixture.

If necessary, softeners can be removed from the method in at least one step, for example by degassing techniques, especially in at least one of steps f) and g). This is especially important for the production of soft capsules having a low softener content and high swelling strength (controlled release capsules).

Compared with WO 01/37817 A1, as a result of the partly crystalline or microcrystalline gel structure, further softeners such as, for example, sorbitol, maltitol or mannitol can be used in smaller fractions so that the sorption behaviour and thus the product properties under changing conditions can be improved.

Admixtures

1. Foreign Nucleating Agents

Foreign nucleating agents can be supplied to the process especially at low softener contents WM₀ in at least one of steps a) to g) in order to facilitate network formation under difficult conditions and increase the network density. They are selected from the following groups:
Nanoparticles: nanoparticles of mono-, oligo- and polysaccharides; microcrystalline cellulose; surface-treated microcrystalline cellulose; polysaccharide microcrystallites; starch microcrystallites; mineral micro- and nanocrystallites such as, for example, boron nitride, sorbitol derivatives, especially 3,4-dimethyl dibenzylidene sorbitol; titanium oxide; calcium carbonate; nanoclays; mixtures of elements of this group.
   2. Nuclei Stabilisers

Nuclei stabilisers can be supplied to the mixture of networking polysaccharides in at least one of steps d) to f) in order to suppress crystallite growth especially in highly concentrated fluids of networking starch. Generally used as nuclei stabilisers are highly branched polysaccharides which show no gel formation or only form very weak gels after days or weeks. Examples are glycogen, amylopectin, or agaropectin. Amylopectins having a blue value of less than 0.08 and/or having an iodine affinity of less than 0.7 g/100 g are preferably used.

3. Additives

Additives can be supplied in at least one of steps a) to g) to improve the workability, to influence the network formation and to modify the product properties having fractions in wt. % of 0.01% to 10%, preferably of 0.02% to 7%, more preferably of 0.03% to 5%. Among others, additives and adjuvants which correspond to the prior art for the manufacture of thermoplastic starch, can also be used for starch gel. Additives are especially selected from the following group of substances:
Food additives, especially antioxidants and food stabilisers; glycerol derivatives; mono-, di- and triglycerides and their stearates; glycerol monostearate; polyglycerol esters, especially of edible fatty acids; mono-, di- or triglycerides of edible fatty acids; polyethylene glycols; polyethylene glycol esters, especially of edible fatty acids; lecithins; non-ionic and ionic wetting agents and tensides; emulsifiers; complexing agents; amylose complexing agents; Na-2-stearoyl lactate; aliphatic alcohols; fatty acids, especially stearic acids, aliphatic and aromatic esters; pyridine; sugar; sugar esters, especially sugar esters of edible fatty acids; fats; fatty acid esters; wax, especially vegetarian wax such as Carnauba wax, Candelilla wax, Japan wax, Ouricury wax, Myrica gale wax, jojoba wax; polyolefin wax; natural resin; shellac; chitin; collagen, casein; mono- and oligosaccharides; dextrans; proteins; peptides; polypeptides, especially plant polypeptides; cellulose, cellulose derivatives, especially hydroxypropylated cellulose; hydrocolloids, especially alginates, carrageenan, galactomannans, glucomannans; dyes; substances usable as foodstuffs; flavourings; mixtures of elements of this group.
   4. Fillers

Fillers can be supplied in at least one of steps a) to g), in order to modify the properties of the material and/or to reduce the specific raw material costs per kilo. Generally eligible are fillers which are used in plastics and bioplastics technology according to the prior art, and these are especially selected from the following group:
Minerals, especially titanium dioxide, talc, clays, wood flour; lignin; fibres, especially natural fibres such as cotton, hemp fibres, flax, ramie, jute fibres; soot; clays; native starch; inhibited starch; cross-linked starch; starch having an amylose content of more than 40%; mixtures of elements of these groups.
   5. Disintegrators

Materials used in galenicals in accordance with the prior art can be used as disintegrators, such as for example, carbonates and hydrogen carbonates of alkali and alkaline earth ions, especially calcium carbonate. In addition, amylases are also eligible. A disintegrator or mixtures of disintegrators can be added in measured quantities in one of steps a) to c) or g).

6. Special Admixtures

The viscosity of the gel can be drastically improved by special admixtures of rubber-like materials, especially hydrocolloids, since the special admixture present as a separate phase in the starch-gel matrix can take up stress peaks. The special admixtures are preferably selected from the following group:
galactomannans such as guar gum or carob bean kernel meal; pectins, especially rhamnogalacturonans and protopectins; dextrans; xanthan; zymosan; hydrocolloids from sea salt, such as alginates, agar agar, agarose, carrageen and carrageenans; furcellaran; hydrocolloids from lichen, such as lichenin and isolichenin, or hydrocolloids as exudates from woods such as tragant (Astragulus gum), Karaya gum, gum arabicum, Kutira gum; inulin; latex; chitin; chitosan; collagen; casein; mixtures of elements of these groups.

In order to obtain optimum results, the finest possible distribution of this phase in the matrix is decisive. For the same fraction of special admixture, the viscosity gain depends decisively on its distribution in the matrix and the particle size. This is made possible on the one hand by the special admixture being pre-prepared as the finest possible powder and on the other hand, by this admixture being preliminarily swollen and then added to the basic starch in the native state with a low softener content. As a result of the shear forces acting during the mixing, the swollen soft particles of the special admixtures are fragmented and ground by the hard native starch grains so that a correspondingly finely distributed phase of the special admixture can be obtained.

The conditions for admixing the special admixtures to obtain a highly disperse phase of the special admixtures are:
A. The special admixtures have a softener content in wt. % at the time of supply of 5-90%, preferably 11-90%, more preferably 18-90%, especially 26-90%, most preferably 33-90%. Water is preferably used as a softener or swelling agent.
B. The average particle size distribution of the special admixtures with a 5% to 20% water content lies in the range 150 mü-0.1 mü, preferably in the range 100 mü-0.1 mü, more preferably in the range 50 mü-0.1 mü, especially in the range 10 mü-0.1 mü, most preferably in the range 5 mü-0.1 mü (1 mü=1 micrometer=1 μm).
C. The special admixtures are added to the basic starch in the native, pregelatinised, partly or completely plasticised state in at least one of steps a) to c), preferably in step a) while the softener content of the basic starch in wt. % at this time lies in the range 1-50%, preferably in the range 1-30%, more preferably in the range 1-20%, especially in the range 1-15%, most preferably in the range 1-12%.

By means of an optimum procedure, a special admixture can be dispersed in the matrix as a highly dispersed phase wherein the average size of this phase lies in the range 50 mü-0.07 mü, preferably in the range 20 mü-0.07 mü, more preferably in the range 7 mü-0.07 mü, especially in the range 3 mü-0.07 mü, most preferably in the range 1 mü-0.07 mü.

The admixing of special admixtures under the specified conditions is also advantageous for the production of TPS soft capsules with improved viscosity in addition to the production of viscous starch-gel soft capsules.

7. Resistant Starches

In order to increase the prebiotic effect of starch-gel-based soft capsules and improve the sorption behaviour, additional resistant starches, preferably in the form of a fine powder can be admixed as an admixture in at least one of steps a) to g). They can be especially selected from the following group:

Resistant starches of the first type, resistant starches of the second type, resistant starches of the third type, starch-gel-based resistant starches, combinations of elements of both groups.

Structural Type of Starch Networks

By means of suitable process control it can be achieved that the forming crystallites at room temperature preferably have an A-structure. Compared with the B-structural type which is stable at room temperature, this structural type exhibits a drastically reduced water absorption for the same air humidity whereby more favourable sorption behaviour is achieved. The A structural type which is metastable at room temperature can be frozen in by kinetic control and thus also obtained at room temperature. A further possibility for obtaining the A structural type is provided by heat treatment wherein the B structural type is converted into the desired A structural type. The required temperature, which must be applied only briefly, lies above 100° C.

Properties of Soft Capsules

The degree of swelling Q (Q=volume after swelling/volume before swelling) of the soft capsule shell conditioned at 50% air humidity and 25° C. on insertion in water at 25° C. in the maximum swollen state lies in the range 1.1-20, preferably in the range 1.1-10, most preferably in the range 1.1-7. For controlled release capsules the degree of swelling Q lies in the range 1.03 to 7, preferably in the range 1.03 to 5, most preferably in the range 1.03 to 3.

The breaking strength of the soft capsule shells conditioned at 50% air humidity and 25° C. lies in the range 1 MPa-30 MPa, preferably in the range 1.5 MPa-20 MPa, most preferably in the range 2 MPa to 17 MPa.

The breaking elongation of the soft capsule shells conditioned at 50% air humidity and 25° C. lies in the range 10-200%, preferably in the range 15% to 150%, most preferably in the range 20-125%.

The total softener content of the soft capsule shells after conditioning at 50% air humidity and 25° C. lies in the range 10-70%, preferably in the range 14-60%, most preferably in the range 18-50%.

Compared with thermoplastic-starch-based soft capsule shells, soft capsule shells according to the proposed method have a flatter sorption curve profile (water content as a function of water activity). Lower water contents can be obtained for the same water activity. This behaviour is especially marked for water activities above 0.5, especially above 0.7.

Parameters

| | |
|---|---|
| T_{L0} | Minimum temperature at which the networking |
| | starches dissolve |
| T_{LR} | Recrystallisation temperature of networking |
| | starches in thermodynamic equilibrium after |
| | dissolving at T_{LO} |
| T_{LÜ} | Overheating temperature T_{LU} > T_{LO} |
| T_{LM} | Temperature at which the metastable state of |
| | the suppressed nuclei growth can be |
| | maintained for 10 seconds |
| delta T_{LU} | Undercooling delta T_{LU} = T_{LR} − T_{LM} |
| T_{L1} | Temperature of the solution when the second |
| | or third fluid is mixed into the first fluid |
| | T_{ML} < T_{L1} < T_{LU}, especially T_{ML} <= T_{L1} < T_{LR} |
| T₁ | Temperature of the first fluid before supply |
| | of the second or third fluid |
| T_{M} | Temperature during the mixing process |
| T₃ | Temperature at the end of the mixing process |
| Tₖ | Temperature at the beginning of network |
| | formation |
| delta t_{d} | Duration of transfer in step d) |
| delta tₑ | Duration of transfer in step e) |
| v_{d} | Heating rate in step d) |
| Ve | Heating rate in step e) |
| Zₖ | Number of nuclei in the third fluid at T_{L1} |
| Z_{N} | Number of foreign nuclei in the mixture |
| | before the first and second or third fluid |
| Z | Number of active nuclei during network |
| | formation |
| C_{PN} | Concentration of networking starch in the |
| | second or third fluid |
| C_{PNM} | Concentration of networking starch in the |
| | mixture |
| C_{Sta} | Concentration of nuclei stabiliser in the |
| | first fluid |
| C_{StaM} | Concentration of nuclei stabiliser in the |
| | mixture |
| C_{N} | Concentration of foreign nucleating agent in |
| | the mixture |
| W_{Md} | Softener content in step d) |
| WM₁ | Softener content of the basic starch at the |
| | beginning of the thermoplastic method |
| WM₂ | Softener content of the mixture after |
| | addition of the second or third fluid |
| WM₃ | Softener content at the end of the mixing |
| | process |
| WM₀ | Softener content during network formation |
| W₀ | Water content during network formation |
| W₁ | Water content after swelling of a film with |
| | W₀ in water |
| G_{d} | Shear velocity in step d) |
| G_{g} | Shear velocity in step g) |
| p_{W} (T) | Water vapour pressure at temperature T |
| N₀/V₀ | Network density after network formation has |
| | been completed |
| DP | Average degree of polymerisation |
| CL | Average chain length (number of monomer units |
| | of unbranched chain segments) |
| Q_{b} | Average degree of branching: number of moles |
| | of branched alpha-glucan units/number of |
| | moles of total alpha glucan units |
| BV | Blue value |
| IA | Iodine affinity [g/100 g] |
| M_{W} | Weight average of molecular weight |
| | distribution |
| T_{g} | Glass transition temperature |

The softener and water contents respectively relate to the basic and networking starches, i.e., to starches which are constituent components of the network. A network containing, for example, 10 g basic starch, 3 g networking starch, 11 g water, 2 g glycerol, 7 g sugar and 5 g of an admixture thus has a softener content WM₀ of 100*(11+2)/(11+2+10+3)=50% and a water content of 100*11/(11+10+3)=45.8%.

### EXAMPLES

Further advantages, features and possible applications of the invention are obtained from the following description of exemplary embodiments which are not to be regarded as restricting.

### Example 1

Formulation

67% (relative to dry weight) basic starch: 70% potato starch (10% water), native, 30% maize starch (10% water), native
19% (relative to dry weight) networking starch: 50% high amylose starch, native (50% amylose content, 15% water), 40% enzymatically debranched tapioca starch (15% water), 10% maltodextrin (15% water)
Admixture: 0.7% lecithin, 0.5% Carnauba wax, 5% calcium carbonate, addition together with basic starches
Special admixtures: 4.8% (relative to dry weight) xanthan (40% water), 3% (relative to dry weight) latex (emulsion), addition before step b)
Softener in step b) 30% water, 5% sorbitol, 5% maltitol (relative to the basic starch, dry weight)
Softener in step d) 75% water, 5% glycerol (relative to networking starches, dry weight)
   Method

Double-screw extruder, metering of networking starches via Sulzer mixer and thermally regulated process sections with shear flow, dissolution of the networking starches together at 190° C. (1 min, 30 bar), pH 9, undercooling to 60° C. (30 sec). Injection of the undercooled solution into the double-screw extruder at a bulk temperature of the basic starch of 130° C., mixing via return kneading elements, following by evacuation, melt pump and wide-slit extrusion, followed by a chill roll section, longitudinal division of the film into two and supply to the rotary die plant, production and filling of the shaped bodies, conditioning.

### Example 2

Modification 1 of example 1: separate solution/undercooling/injection of high-amylose starch (HAS) and debranched tapioca starch (TAS),
Softener in step d1) for HAS: 75% water
Softener in step d2) for TAS: 70%, 10% glycerol
HAS: dissolution at 190° C. (1 min, 30 bar, pH 9), under cooling to 80° C. (20 sec)
TAS: dissolution at 200° C. (1 min, 20 bar, pH 7), under cooling to 50° C. (20 sec)

### Example 3

Modification 2 of example 1: formulation identical, but plasticisation of the basic starches and working in the admixtures and the special admixtures preliminarily in one step a) using double-screw extruder, followed by granulation. Re-plasticisation of the granules in a single-screw extruder, dissolution/undercooling/injection of the networking starches in the single-screw extruder as in modification 1 of example 1, mixing using Madoc element, no melt pump, the other process steps are identical to Example 1.

1-19. (canceled)

20. A method for the production of starch-gel-based shaped bodies, comprising forming a gel from a total mixture of components comprising at least one basic starch and at least one networking starch by homocrystallisation and heterocrystallisation, wherein the basic starch and networking starch are prepared separately and individually.

21. The method according to claim 20, wherein the basic starch is plasticised, the networking starch is dissolved and the components are mixed in a molecular disperse fashion.

22. The method according to claim 21, wherein the basic starch is plasticized after the addition of the networking starch.

23. The method according to claim 22, wherein at least a portion of the gel formation occurs before forming of the total mixture into a resulting shaped bodies.

24. The method according to claim 20, wherein the method comprises the following steps in at least one process zone:
a) Supplying respectively one basic starch;
b) Action of respectively one first softener on the respectively one basic starch;
c) Transferring the respectively one basic starch into respectively one first fluid wherein respectively one first mixture is formed;
d) Transferring respectively one networking starch into respectively one second fluid by the action of respectively one second softener;
e) Transferring the respective second fluid into a respective third fluid;
f) Incorporating the respective second fluid from step d) and/or the respective third fluid from step e) into one of the respective first mixtures from steps a) to c);
g) Combining the respective mixtures from steps a) to f) into at least one preferably molecular disperse total mixture;
h) Forming at least one film from the at least one total mixture formed in step g);
i) Supplying the at least one film formed in step h) to a reforming plant and production of resulting shaped bodies from the at least one film, especially supplying the at least one film formed in step h) to a continuous encapsulating plant, for example, a rotary die plant, and production of heat-sealed soft capsules containing filler or active ingredient;
j) Initiating the formation of a starch network from the at least one total mixture formed in step g), especially by homocrystallisation among one another between respective macromolecules of the respectively at least one networking starch and/or by heterocrystallisation between these respective macromolecules and respective macromolecules of the respectively at least one basic starch, after steps a) to h) or a) to i) have been completed.
k) Setting the desired softener or water content of the resulting shaped bodies, especially the soft capsules by conditioning under a prepared temperature and air humidity profile.

25. The method according to claim 24, wherein in at least one of steps d) to g), especially after step f) and before step h), a softener is removed at least partly actively from the method by evacuation techniques.

26. The method according to claim 24, wherein, in step d) the second fluid containing networking starch is overheated and in step e) the third fluid is undercooled if necessary.

27. The method according to claim 24, wherein in at least one of steps a) to g) a foreign nucleating agent is supplied at least once and/or the second or third fluid is treated with ultrasound during or after step e).

28. The method according to claim 24, wherein at least one additive is supplied to the method in at least one of steps a) to g).

29. The method according to claim 24, wherein in at least one of steps a) to g), preferably in one of steps a) to c) a special additive is added and the resulting starch gel having high viscosity contains this special additive in the form of a highly disperse phase, wherein the average size of this phase is in the range 50 mü-0.07 mü, preferably in the range 20 mü-0.07 mü, more preferably in the range 7 mü-0.07 mü, especially in the range 3 mü-0.07 mü, most preferably in the range 1 mü-0.07 mü.

30. The method according to claim 24, wherein a film formed in step h) is divided in the longitudinal direction into two halves which are fed separately to a rotary die plant, especially over the same distance and at the same speed, so that the two halves of the heat-sealed soft capsules have undergone an identical pre-history, i.e., have been formed simultaneously and in parallel into a film in step h).

31. The method according to claim 24, wherein the steps a) to h) are carried out in parallel in two different process zones and in the rotary die method the two halves of the heat-sealed soft capsules are made of two films which originate from the two different process zones.

32. A shaped body comprising a soft capsule wherein the soft capsule is produced by the method according to claim 24.

33. The soft capsule according to claim 32, wherein the soft capsule is used for controlled release applications.

34. The soft capsule according to claim 32, comprises a multilayer shaped body, wherein at least one layer of the shaped body comprises the starch gel.

35. A soft capsule according to claim 32, wherein the soft capsule shell is in the form of a film of maximum 0.3 mm thickness, the film has been dried and brought in this state into an atmosphere with a maximum 43% air humidity at room temperature and stored there until the weight of the film no longer varied and the film had a water content of W₄₃, and after the film had then been brought into an atmosphere having at least 90% air humidity at room temperature until the weight no longer varied and the film had acquired a water content of W₉₀, the difference in the water content in wt. % is 3%-25%, preferably 3%-20%, more preferably 3%-17%, especially 3%-13%, most preferably 3%-10%, most especially 3%-7%.

36. The soft capsule shell according to claim 32, wherein a crystalline fraction obtained by separating the crystalline fraction from an amorphous fraction of a wide-angle x-ray diffraction curve of a sample having 7 to 14 wt. % water, between the scattering angles 3°<2-theta<37°, is 15%-100%.

37. The soft capsule shell according to claim 32, wherein a crystalline fraction obtained by separating the crystalline fraction from an amorphous fraction of a wide-angle x-ray diffraction curve of a sample having 7 to 14 wt. % water, between the scattering angles 3°<2-theta<25%-100%.

38. The soft capsule shell according to claim 32, wherein a crystalline fraction obtained by separating the crystalline fraction from an amorphous fraction of a wide-angle x-ray diffraction curve of a sample having 7 to 14 wt. % water, between the scattering angles 3°<2-theta<35%-100%.

39. The soft capsule shell according to claim 32, wherein a crystalline fraction obtained by separating the crystalline fraction from an amorphous fraction of a wide-angle x-ray diffraction curve of a sample having 7 to 14 wt. % water, between the scattering angles 3°<2-theta<45%-100%.

40. The soft capsule shell according to claim 32, wherein a crystalline fraction obtained by separating the crystalline fraction from an amorphous fraction of a wide-angle x-ray diffraction curve of a sample having 7 to 14 wt. % water, between the scattering angles 3°<2-theta<60%-100%.

41. The soft capsule according to claim 32, wherein the soft-capsule shell consists of a single-phase transparent starch gel produced by a rotary die method.

42. The soft capsule according to claim 32, wherein the soft-capsule shell contains a resistant starch in the form of an additive, having a fraction in wt. % of 1%-70%.

43. The soft capsule according to claim 32, wherein the soft-capsule shell contains a resistant starch in the form of an additive, having a fraction in wt. % of 3%-50%.

44. The soft capsule according to claim 32, wherein the soft-capsule shell contains a resistant starch in the form of an additive, having a fraction in wt. % of 5%-45%.

45. The soft capsule according to claim 32, wherein the soft-capsule shell has a prebiotic effect and, compared with a TPS soft capsule shell with a comparable softener content, a glyceamic index of 10%-95% lower.

46. The soft capsule according to claim 32, wherein the soft-capsule shell has a prebiotic effect and, compared with a TPS soft capsule shell with a comparable softener content, a glyceamic index of 20%-95% lower.

47. The soft capsule according to claim 32, wherein the soft-capsule shell has a prebiotic effect and, compared with a TPS soft capsule shell with a comparable softener content, a glyceamic index of 30%-95% lower.
